(19) United States
(12) Patent Application Publication
(10) Pub. No.: US 2009/0274795 A1
(43) Pub. Date: Nov. 5, 2009

(54) Enzyme Granulate l Containing Phytase

(75) Inventors: Markus Lohscheidt, Heidelberg (DE); Roland Betz, Niederkirchen (DE); Jörg Braun, Essingen (DE); Wolf Pelletier, Ottersheim (DE); Peter Ader, Heppenheim (DE)

Correspondence Address: CONNOLLY BOVE LODGE & HUTZ, LLP, P O BOX 2207, WILMINGTON, DE, 19899, US

(73) Assignee: BASF SE, LUDWIGSHAFEN (DE)

(21) Appl. No.: 11/991,837

(22) PCT Filed: Sep. 11, 2006
(86) PCT No.: PCT/EP2006/066218

§ 371 Date: Mar. 11, 2008

(30) Foreign Application Priority Data
Sep. 12, 2005 (DE) 10 2005 043 323.5

(51) Int. Cl. C12N 9/16; A23L 1/30

(52) U.S. Cl. 426/61; 435/196

(57) ABSTRACT

The present invention relates to novel phytase-comprising enzyme granules, those particles of which have
A) a core which comprises at least one phytase and at least one solid carrier material suitable for feeds, and
b) a coating arranged on the core,
   the cores of the particles or the entire particle, after if appropriate required grinding, on suspension or dissolution in demineralized water at 25° C. giving a pH in the range from 4.5 to 6.5, preferably in the range from 4.6 to 6.0, and particularly preferably from 4.7 to 5.5 and which are suitable as feed additives, and also a method for production thereof. The invention also relates to the use of the phytase-comprising enzyme granules in feed compositions and, in particular, pelleted feed compositions, which are obtainable using the phytase-comprising enzyme granules.

The present invention relates to novel phytase-comprising enzyme granules which are suitable as feed additives, and also to a method for production thereof. The invention also relates to the use of the phytase-comprising enzyme granules in feed compositions and, in particular, pelleted feed compositions, which are obtainable using the phytase-comprising enzyme granules.

It is generally customary to add phytase to animal feed in order to ensure better feed utilization, better product quality or lower pollution of the environment. In addition, it is current practice to supply animal feeds in pelleted form, since pelleting not only facilitates feed intake, but also improves handling of the feedstuff. In addition, it has been found that in the case of pelleted feedstuff, certain feed components are digested better, and ingredients added to the feedstuff such as, for example, vitamins, enzymes, trace elements, can be better incorporated in the feed mixture.

To reduce the microbial loading (sanitation) of such animal feeds, heat treating is frequently carried out. A heat treatment also proceeds in the context of the conditioning required for pelleting, in which the feedstuff is admixed with steam and thereby heated and moistened. In the actual pelleting step, the feedstuff is forced through a matrix. Other processes used in the feed industry are extrusion and expansion. The action of heat in all of these processes is a problem, since the enzymes such as phytase present in such feed mixtures are generally thermally unstable. Therefore, various efforts have been made to improve the thermal stability and, in particular, the pelleting stability of enzyme-comprising feed compositions.

There have been various proposals to improve the pelleting stability of phytase-comprising enzyme granules by coating uncoated enzyme-comprising granules.

WO 2000/47060 describes, for example, phytase-comprising enzyme granules which are suitable as feed additives and which have a polyethylene glycol coating.

WO 01/00042 teaches a method for coating phytase-comprising enzyme granules with polymers. Coating agents which are proposed are aqueous solutions of polyalkylene oxide polymers, of homo- and copolymers of vinylpyrrolidone, of polyvinyl alcohols, and of hydroxypropylmethylcellulose, and also aqueous dispersions of alkyl (meth)acrylate polymers and polyvinyl acetate dispersions.

WO 03/059086 in turn teaches a method for producing phytase-comprising enzyme granules having improved pelleting stability, in which phytase-comprising raw granules are coated with an aqueous dispersion of a hydrophobic substance.

By means of the coating, the stability of the granules against decreasing phytase activity can be basically improved, but the stabilities achieved are not completely satisfactory.

EP-A-0 257 996 proposes stabilizing enzymes for feed mixtures by pelleting them in a mixture with a carrier which has a main fraction of cereal flour.

WO 98/54980 in turn describes enzyme-comprising granules having improved pelleting stability which are produced by extruding an aqueous enzyme solution with a carrier based on an edible carbohydrate, and subsequent drying. Coating the granules is not described. The stability of these granules is not satisfactory.

PCT/EP 05/000826 in turn discloses improving the stability of the enzyme in liquid or solid enzyme formulations by adding gum Arabic or a plant protein to these.

It is therefore an object of the present invention to provide phytase-comprising enzyme granules having improved enzyme stability, in particular pelleting stability. The enzyme granules should, in addition, be able to be produced in a simple manner and inexpensively. In addition, no losses in enzyme activity should occur yet during the production.

It has surprisingly been found that phytase-comprising enzyme granules exhibit particularly good pelleting stability when these are coated granules, the particles of which, in addition to at least one coating, comprise a phytase-comprising core which comprises a solid carrier material suitable for feeds and the cores of the particles or the entire particle, after if appropriate required grinding, on suspension or dissolution in demineralized water at 25° C. give a pH in the range from 4.5 to 6.5, preferably in the range from 4.6 to 6.0, and particularly preferably from 4.7 to 5.5.

The invention accordingly relates to a phytase-comprising enzyme granule for feeds, the particles of which have
A) a core which comprises at least one phytase and at least one solid carrier material suitable for feeds, and
b) a coating arranged on the core,
   the cores of the particles or the entire particle, after if appropriate required grinding, on suspension or dissolution in demineralized water at 25° C. giving a pH in the range from 4.5 to 6.5, preferably in the range from 4.6 to 6.0, and particularly preferably from 4.7 to 5.5.

The inventive enzyme granules are distinguished by a particularly high stability, in particular a particularly high pelleting stability, and may be produced in a simple manner, the loss in enzyme activity during production being comparable to the loss in enzyme activity in comparable methods of the prior art. Accordingly, the present invention also relates to the production method described here and to the use of the inventive enzyme granules in feed compositions, especially in pelleted feed compositions.

The phytase-comprising granule particles of the inventive enzyme granules have a core and at least one hydrophobic coating arranged on the surface of the core, the core comprising at least one phytase and at least one solid carrier material suitable for feeds.

According to the invention, the cores of the particles or the entire particle, if appropriate after grinding, on suspension or dissolution in demineralized water at 25° C., give a pH in the range from 4.5 to 6.5, preferably in the range from 4.6 to 6.0, and particularly preferably in the range from 4.7 to 5.5. Generally, to determine the pH, 5 g of the uncoated cores or coated cores are dissolved at 25° C. in 200 ml of demineralized water and the pH established after 30 min is determined using a glass electrode or a pH measuring instrument.

According to a preferred embodiment of the invention, the core-forming substances, in addition to the phytase and the solid carrier material, comprise at least one agent for setting a pH of 4.5 to 6.5, preferably 4.6 to 6.0, and particularly preferably of 4.7 to 5.5, for example a buffer or a base, the latter, in particular, when the core-forming materials themselves have acid groups.

Suitable substances for setting the pH are sufficiently known to those skilled in the art, for example from Küster-Thiel, Rechentafeln für die chemische Analytik [Calculation tables for chemical analysis], 102nd edition, 1982, Walter de Gruyter-Verlag and Handbook of Chemistry and Physics, 76th ed. 1995-1996, CRS Press 8-38 ff.; DIN Normenheft 22, Richtlinien für die pH-Messung in industriellen Anlagen [Guidelines for pH measurement in industrial plants], Berlin: Beuth 1974; DIN 19266 (August 1979); DIN 19267 (August 1978); Naturwissenschaften 65, 438 ff. (1978). Kontakte (Merck) 1981, No. 1, 37-43.

Examples of suitable buffers are acetate, propionate, tartrate, hydrogencarbonate, phthalate, hydrogenphthalate, in particular the sodium, potassium or calcium salts of the abovementioned substances, including their hydrates or dihydrates, phosphate buffer, potassium or sodium phosphate, their hydrates or dihydrates, sodium or potassium carbonate. Examples of suitable bases are sodium or potassium carbonate, sodium, potassium, calcium, magnesium, ammonium hydroxide, or ammonia water or oxides thereof.

The amount of buffer or base is typically in the range from 0.1 to 5% by weight, based on the total weight of the core-forming nonaqueous components. In principle there need be no addition of buffer when the components of the core material in the composition present in the core already give such a pH. In particular, a core material has proven useful which is obtainable by a method in which, to produce the core, use is made of an aqueous enzyme concentrate which, at 25° C., has a pH in the range from 4.5 to 6.5, preferably 4.6 to 6, and particularly preferably from 4.7 to 5.5. In this case the pH of the enzyme concentrate is determined directly using a glass electrode or a pH measuring instrument.

In addition, the core-forming material, according to the invention, comprises at least one solid carrier material suitable for feeds. The carrier material typically makes up at least 50% by weight, in particular at least 55% by weight, and frequently at least 60% by weight, of the nonaqueous components of the core material, e.g. 50 to 96.9% by weight, preferably 55 to 94.8% by weight, and in particular 60 to 89.7% by weight, based on the nonaqueous components of the core.

As feed-compatible carrier materials, use can be made of customary inert inorganic or organic carriers. An “inert” carrier must not exhibit any adverse interactions with the enzyme(s) of the inventive feed additive, such as, for example, cause irreversible inhibition of the enzyme activity, and must be harmless for use as an auxiliary in feed additives. Examples of suitable carrier materials which may be mentioned are: low-molecular-weight organic compounds, and also higher-molecular-weight organic compounds of natural or synthetic origin, and also inert inorganic salts. Preference is given to organic carrier materials. Among these, carbohydrates are particularly preferred.

Examples of suitable low-molecular-weight organic carriers are, in particular, sugars such as, for example, glucose, fructose, sucrose. Examples of higher-molecular-weight organic carriers which may be mentioned are carbohydrate polymers, in particular those which comprise α-D-glucopyranose, amylose or amylopectin units, in particular native and modified starches, microcrystalline cellulose, but also α-glucans and β-glucans, pectin (including protopectin) and glycogen. Preferably, the carrier material comprises at least one water-insoluble polymeric carbohydrate, in particular a native starch material such as, in particular, corn starch, rice starch, wheat starch, potato starch, starches of other plant sources such as starch from tapioca, cassaya, sago, rye, oats, barley, sweet potatoes, arrowroot and the like, in addition cereal flours such as, for example, corn flour, wheat flour, rye flour, barley flour and oat flour, and also rice flour. Suitable materials are, in particular, also mixtures of the abovementioned carrier materials, in particular mixtures which predominantly, i.e. at least 50% by weight, based on the carrier material, comprise one or more starch materials. Preferably, the water-insoluble carbohydrate makes up at least 50% by weight, in particular at least 65% by weight, and especially at least 80% by weight, of the carrier material. Particularly preferred carrier materials are starches which comprise no more than 5% by weight, and in particular no more than 2% by weight, of protein or other components. A further preferred carrier material is microcrystalline cellulose. This can be used alone or in a mixture with the abovementioned carrier materials. If the microcrystalline cellulose is used in a mixture with other carrier materials, it preferably makes up no more than 50% by weight, in particular no more than 30% by weight, for example 1 to 50% by weight, in particular 1 to 30% by weight, and especially 1 to 10% by weight, of the carrier material.

Inorganic carrier materials which come into consideration are in principle all inorganic carrier materials known for feeds and feed additives, for example inert inorganic salts, for example sulfates or carbonates of alkali and alkaline earth metals such as sodium, magnesium, calcium and potassium sulfate or carbonate, in addition feed-compatible silicates such as talcum and silicic acids. The amount of inorganic carrier material, based on the total amount of carrier material, will generally not exceed 50% by weight, particularly 35% by weight, and very particularly 20% by weight. In a preferred embodiment, the organic carrier materials make up the total amount or virtually the total amount, that is at least 95% by weight, of the carrier material.

In addition, the enzyme core comprises at least one phytase, mixtures of different phytases or mixtures of phytase with one or more other enzymes also being able to be present. Typical enzymes for feeds are, in addition to phytase, for example oxidoreductases, transferases, lyases, isomerases, ligases, lipases, and in particular hydrolases different from phytase. Examples of hydrolases, that is enzymes which cause a hydrolytic cleavage of chemical bonds, are esterases, glycosidases, keratinases, ether hydrolases, proteases, amidases, aminidases, nitrilases, and phosphatases. Glycosidases (EC 3.2.1, also termed carbohydrases) comprise not only endo- but also exoglycosidases, which cleave not only α- but also β-glycosidic bonds. Typical examples thereof are amylases, maltases, cellulases, endoxylanases, for example endo-1,4-β-xylanase or xylan endo-1,3-β-xylosidase, β-glucanases, in particular endo-1,4-β- and endo-1,3-β-glucanases, mannanases, lysozymes, galactosidases, pectinases, β-glucuronidases and the like.

The expression “phytase” comprises not only natural phytase, but also any other enzyme which exhibits phytase activity, for example is capable of catalyzing a reaction which liberates the phosphorus or phosphate from myoinositol phosphates. The phytase can be not only a 3-phytase (EC 3.1.3.8) but also a 4- or 6-phytase (EC 3.1.3.26) or a 5-phytase (EC 3.1.3.72) or a mixture thereof. Preferably, the phytase belongs to the enzyme class EC 3.1.3.8.

The phytase used according to the invention is not subject to any restrictions and can be not only of microbiological origin, but also a phytase obtained by genetic modification of a naturally occurring phytase, or by de-novo construction. The phytase can be a phytase from plants, from fungi, from bacteria, or a phytase produced by yeasts. Preference is given to phytases from microbiological sources such as bacteria, yeasts or fungi. However, they can also be of plant origin. In a preferred embodiment, the phytase is a phytase from a fungal strain, in particular from an Aspergillus strain, for example Aspergillus niger, Aspergillus oryzae, Aspergillus ficuum, Aspergillus awamori, Aspergillus fumigatus, Aspergillus nidulans or Aspergillus terreus. Particular preference is given to phytases which are derived from a strain of Aspergillus niger or a strain of Aspergillus oryzae. In another preferred embodiment, the phytase is derived from a bacterial strain, in particular a Bacillus strain, an E. coli strain or a Pseudomonas strain, among these phytases being preferred which are derived from a Bacillus subtilis strain. In another preferred embodiment, the phytase is derived from a yeast, in particular a Kluveromyces strain or a Saccharomyces strain, among these phytases being preferred which are derived from a strain of Saccharomyces cerevisiae. In this invention, the expression “an enzyme derived from phytase” comprises the phytase naturally produced by the respective strain which is either obtained from the strain, or which is coded for by a DNA sequence isolated from the strain and is produced by a host organism which has been transformed using this DNA sequence. The phytase can be obtained from the respective microorganism by known techniques which typically comprise fermentation of the phytase-producing microorganism in a suitable nutrient medium (see, for example, ATCC catalog) and subsequently obtaining the phytase from the fermentation medium by standard techniques. Examples of phytases and of methods for preparing and isolating phytases may be found in EP-A 420358, EP-A 684313, EP-A 897010, EP-A 897985, EP-A 10420358, WO 94/03072, WO 98/54980, WO 98/55599, WO 99/49022, WO 00/43503, WO 03/102174, the contents of which are hereby explicitly incorporated by reference.

The amount of phytase in the core naturally depends on the desired activity of the enzyme granules and the activity of the enzyme used and is typically in the range from 3 to 49.9% by weight, frequently in the range from 5 to 49.7% by weight, in particular in the range from 10 to 44.5% by weight, and especially in the range from 10 to 39% by weight, calculated as dry mass and based on the total weight of all nonaqueous components of the core material.

In a preferred embodiment, the components forming the core, in addition to the feed-compatible carrier material, comprise at least one water-soluble polymer. This polymer acts as binder and at the same time increases the pelleting stability. Preferred water-soluble polymers exhibit a number-average molecular weight in the range from 5×10³ to 5×10⁶ dalton, in particular in the range from 1×10⁴ to 10⁶ dalton. The polymers are water-soluble when at least 3 g of polymer may be dissolved completely in 1 liter of water at 20° C.

The water-soluble polymers used according to the invention comprise
polysaccharides, for example water-soluble modified starches generally having adhesive properties, for example starch breakdown products (dextrins) such as acid dextrins, pyrodextrins, enzymatic partial hydrolyzates (limited dextrins), oxidatively broken down starches and their reaction products of dextrins with cationic or anionic polymers, reaction products of dextrins with octenyl succinate anhydride (OSA), starch-based adhesive, in addition chitin, chitosan, carragheen, alginates, arabic acid salts, gums, e.g. gum Arabic, tragacanth, karaya gum; xanthan gum and gellan gum; galactomannans; water-soluble cellulose derivatives, for example methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose, and also carboxymethylcellulose (CMC);
water-soluble proteins, e.g. proteins of animal origin such as gelatin, casein, in particular sodium caseinate and plant proteins such as soy protein, pea protein, bean protein, rapeseed protein, sunflower protein, cottonseed protein, potato protein, lupin, zein, wheat protein, corn protein and rice protein,
synthetic polymers, for example polyethylene glycol, polyvinyl alcohol and, in particular, the kollidon brands of BASF, vinyl alcohol/vinyl ester copolymers, homo- and copolymers of vinylpyrrolidone with vinyl acetate and/or C₁-C₄-alkyl acrylates,
and biopolymers modified if appropriate, e.g. lignin or polylactide.

Preferred water-soluble polymers are neutral, that is they have no acidic or basic groups. Among these, polyvinyl alcohols, including partially saponified polyvinyl acetates having a degree of saponification of at least 80%, and also, in particular, water-soluble, neutral cellulose ethers such as methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose are particularly preferred.

In a preferred embodiment of the invention, the water-soluble polymer is selected from neutral cellulose ethers. Examples of inventively preferred water-soluble neutral cellulose ethers are methylcellulose, ethylcellulose and hydroxyalkylcelluloses, for example hydroxyethylcellulose (HEC), hydroxyethyl methylcellulose (HEMC), ethyl hydroxyethylcellulose (EHEC), hydroxypropylcellulose (HPC), hydroxypropyl methylcellulose (HPMC) and hydroxybutylcellulose. Among these, methylcellulose, ethylcellulose and mixed cellulose ethers having methyl groups or ethyl groups and hydroxyalkyl groups such as HEMC, EHEC and HPMC are particularly preferred. Preferred methyl- or ethyl-substituted cellulose ethers have a degree of substitution DS (with respect to the alkyl groups) in the range from 0.8 to 2.2 and, in the case of mixed cellulose ethers, a degree of substitution DS with respect to the alkyl groups in the range from 0.5 to 2.0, and a degree of substitution HS with respect to the hydroxyalkyl groups in the range from 0.02 to 1.0.

The fraction of water-soluble polymers is preferably in the range from 0.2 to 10% by weight, in particular 0.3 to 5% by weight, and especially 0.5 to 3% by weight, based on the dough-forming nonaqueous components and is accordingly in these amounts a component of the enzyme-comprising raw granules.

In addition, the core-forming material can additionally comprise a salt stabilizing the enzyme. Stabilizing salts are typically salts of divalent cations, in particular salts of calcium, magnesium or zinc, and also salts of monovalent cations, in particular sodium or potassium, for example the sulfates, carbonates, hydrogencarbonates and phosphates including hydrogenphosphates and ammonium hydrogenphosphates of these metals. Preferred salts are sulfates. Particular preference is given to magnesium sulfate and zinc sulfate, including their hydrates. The amount of salt is preferably in the range from 0.1 to 10% by weight, in particular in the range from 0.2 to 5% by weight, and especially in the range from 0.3 to 3% by weight, based on the total weight of all nonaqueous components of the core material.

The particles of the inventive enzyme granules in addition have at least one coating arranged on the core of the particles. The coating will preferably at least 80% (mean value) and particularly completely cover the surface of the cores.

The weight ratio of core to coating is generally in the range from 70:30 to 99:1, preferably in the range from 75:25 to 98:2, in particular in the range from 80:20 to 96:4, and especially in the range from 85:15 to 95:5.

Suitable coatings are in principle all types of coatings which are known for enzyme granules from the prior art. Preference is given to hydrophobic coatings, that is coatings whose components are water-insoluble or of only limited water solubility. Accordingly, an inventively preferred embodiment relates to phytase-comprising enzyme granules whose particles have a coating at least 90% by weight of which comprises water-insoluble hydrophobic substances.

Hydrophobic materials which come into consideration for the hydrophobic coating are not only polymeric substances but also oligomeric or low-molecular-weight substances. According to the invention, the hydrophobic materials have a high hydrocarbon fraction, the fraction of carbon and hydrogen generally making up at least 80% by weight, in particular at least 85% by weight, of the hydrophobic material. Preference is given to those substances which have a melting point above 30° C., more preferably above 40° C., in particular above 45° C., and especially above 50° C., or in the case of non-melting substances are solid at these temperatures or have a glass transition temperature above these temperatures. Preference is given to hydrophobic materials having melting points in the range from 40 to 95° C., in particular in the range from 45 to 80° C., and particularly preferably in the range from 50 to 70° C.

Preferably, the hydrophobic material is low-acid, and has an acid value less than 80, in particular less than 30, and especially less than 10 (determined as specified in ISO 660).

Examples of hydrophobic materials suitable according to the invention are
polyolefins such as polyethylene, polypropylene and polybutenes;
saturated fatty acids preferably having 10 to 32 carbon atoms, frequently 12 to 24 carbon atoms, and in particular 16 to 22 carbon atoms;
esters of saturated fatty acids, preferably mono-, di- and triglycerides and also esters of saturated fatty acids with fatty alcohols. The fatty alcohols for example have 10 to 32 carbon atoms, in particular 16 to 24 carbon atoms, such as cetyl alcohol or stearyl alcohol. The fatty acids and the fatty acid radicals in the esters of fatty acids preferably have 10 to 32, frequently 12 to 24, carbon atoms and, in particular, 16 to 22 carbon atoms;
waxes, in particular plant waxes and waxes of animal origin, but also montan waxes and montan ester waxes;
polyvinyl acetates;
C₁-C₁₀-alkyl (meth)acrylate polymers and copolymers, preferably those having a number-average molecular weight of from about 100 000 to 1 000 000; in particular ethyl acrylate/methyl methacrylate copolymers and methyl acrylate/ethyl acrylate copolymers.

In a preferred embodiment, the coating-forming material comprises up to at least 70% by weight, particularly up to at least 80% by weight, in particular up to at least 90% by weight, of at least one substance selected from saturated fatty acids, esters of fatty acids and mixtures thereof, esters of fatty acids and, in particular, triglycerides being preferred. Saturated means that the hydrophobic material is essentially free from unsaturated components and correspondingly has an iodine value less than 5 and, in particular, less than 2 (method according to Wijs, DIN 53 241).

Particularly preferably, the coating comprises up to at least 70% by weight, in particular at least 80% by weight, and especially at least 90% by weight, of the above-mentioned triglycerides.

In a preferred embodiment of the invention, the coating agent predominantly, that is up to at least 70% by weight, in particular at least 80% by weight, and especially greater than 90% by weight, comprises hydrogenated vegetable oils, in particular triglycerides of plant origin, for example hydrogenated cottonseed, corn, peanut, soybean, palm, palm kernel, babassu, rapeseed, sunflower and safflower oils. Hydrogenated vegetable oils which are particularly preferred among these are hydrogenated palm oil, cottonseed oil and soybean oil. The most preferred hydrogenated vegetable oil is hydrogenated soybean oil. Similarly, other fats and waxes originating from plants and animals are also suitable, for example beef tallow. Suitable materials are also nature-identical fats and waxes, that is synthetic waxes and fats having a composition which predominantly corresponds to that of the natural products.

The table below mentions some examples of coating materials which are suitable according to the invention:

| | | Melting | |
|---|---|---|---|
| Name | Composition | range | CAS No./INCI |
| Cutina CP from | synthetic cetyl | 46-51° C. | 95912-87-1 |
| Cognis | palmitate | | cetyl palmitate |
| Edenor NHTI-G | Triglyceride | 56-60° C. | 67701-27-3* |
| from Cognis | | | |
| Edenor NHTI-V from | Triglyceride | 57-60° C. | 67701-27-3* |
| Cognis | | | EINECS 266-945-8 |
| Edenor C1892 from | Stearic acid, C16-18 | 66-99° C. | |
| Cognis | | | |
| Edenor HPA from | Fatty acids, palm oil, | 55-57° C. | |
| Cognis | hydrogenated, C16-18 | | |
| Edenor HRAGW | Fatty acids, C16-22 | 64-66° C. | |
| from Cognis | | | |
| Edenor C2285R | Fatty acids, C18-22 | 75-78° C. | 68002-88-0* |
| from Cognis | | | |
| Rilanit from Cognis | Triglyceride | 83-90° C. | |
| Japan wax | principally glycerol | 49-55° C. | rhus succedanea |
| substitute Kahl - | palmitate | | |
| Wachsraffinerie | | | |
| Tefacid from | palm kernel oil Tefacid | 65° C. | 57-10-3 |
| Karlshamns | Palmic 90 | | |
| Soybean fat powder | | 65-70° C. | |
| from Sankyu/Japan | | | |

Suitable products are also those of the company Aarhus Olie, Denmark, marketed under the trademark Vegeol PR, for example Vegeol® PR 267, PR 272, PR 273, PR 274, PR 275, PR 276, PR 277, PR 278 and PR 279.

Waxes suitable as coating materials are, in particular, waxes of animal origin such as beeswax and lanolin, waxes of plant origin such as candelilla wax, carnauba wax, cane sugar wax, caranday wax, raffia wax, Columbia wax, esparto wax, alfalfa wax, bamboo wax, hemp wax, Douglas fir wax, cork wax, sisal wax, flax wax, cotton wax, dammar wax, cereal wax, rice wax, ocatilla wax, oleander wax, montan waxes, montan ester waxes, polyethylene waxes, in addition the products of Suddeutsche Emulsions-Chemie marketed under the trademarks Wukonil, Südranol, Lubranil or Mikronil, or the BASF products having the trademarks Poligen WE1, WE3, WE4, WE6, WE7, WE8 BW, WE9.

Suitable hydrophobic coating materials are, in addition, the following polyolefins: polyisoprene, medium- and high-molecular-weight polyisobutene and polybutadiene. In preferred alkyl (meth)acrylate polymers and copolymers, the alkyl group has 1 to 4 carbon atoms. As specific examples of suitable copolymers, mention may be made of: ethyl acrylate/methyl methacrylate copolymers, which are marketed, for example, under the trademarks Kollicoat EMM 30D by BASF AG, or under the trademark Eudragit NE 30 D by Degussa; and also methacrylate/ethyl acrylate copolymers as are marketed, for example, under the trademark Kollicoat MAE 30DP by BASF AG, or under the trademark Eudragit 30/55 by Degussa in the form of an aqueous dispersion.

Examples of polyvinyl acetate dispersions which may be mentioned are those which are stabilized by polyvinylpyrrolidone and are marketed, for example, under the trademark Kollicoat SR 30D by BASF AG (solids content of the dispersion about 20 to 30% by weight).

In another embodiment of the invention, the coating comprises polymeric substances which have in water an at least limited solubility. Examples of these are
a) polyalkylene glycols, in particular polyethylene glycols, preferably those having a number-average molecular weight of from about 400 to 15 000, such as, for example, about 400 to 10 000;
b) polyalkylene oxide polymers or copolymers, preferably those having a number-average molecular weight of from about 4000 to 20 000, such as, for example, about 7700 to 14 600; in particular block copolymers of polyoxyethylene and polyoxypropylene;
c) polyvinylpyrrolidone, preferably having a number-average molecular weight of from about 7000 to 1 000 000, such as, for example, about 44 000 to 54 000;
d) vinylpyrrolidone/vinyl acetate copolymers, preferably those having a number-average molecular weight of from about 30 000 to 100 000, such as, for example, about 45 000 to 70 000;
e) polyvinyl alcohols, preferably those having a number-average molecular weight of from about 10 000 to 200 000, such as, for example, about 20 000 to 100 000;
f) modified celluloses and cellulose derivatives, such as, for example, hydroxypropyl methylcellulose, preferably having a number-average molecular weight of from about 6000 to 80 000, such as, for example, about 12 000 to 65 000; or else methylcellulose, ethylcellulose and hydroxyalkylcelluloses such as, for example, hydroxyethylcellulose (HEC), hydroxyethyl methylcellulose (HEMC), ethyl hydroxyethylcellulose (EHEC), hydroxypropylcellulose (H PC) and
g) polyvinyl alcohol-polyethylene glycol graft copolymers;
h) modified starches (for example reaction products of octenyl succinate anhydride (OSA) and starch).

Examples of suitable polyalkylene glycols a) which may be mentioned are: polypropylene glycols and, in particular, polyethylene glycols of a different molar mass, such as, for example, PEG 4000 or PEG 6000, obtainable from BASF AG under the trademarks Lutrol® E 4000 and Lutrol® E 6000, and also the Kollidon brands from BASF.

Examples of the above polymers b) which may be mentioned are: polyethylene oxides and polypropylene oxides, ethylene oxide/propylene oxide mixed polymers and also block copolymers, made up from polyethylene oxide and polypropylene oxide blocks, such as, for example, polymers which are obtainable from BASF AG under the trademark Lutrol® F68 and Lutrol® F127.

Examples of the above polymers c) which may be mentioned are: polyvinylpyrrolidones, as are marketed, for example, by BASF AG under the trademark Kollidon® or Luviskol®.

An example of the abovementioned polymers d) which may be mentioned is: a vinylpyrrolidone/vinyl acetate copolymer which is marketed by BASF AG under the trademark Kollidon® VA64.

Examples of the above polymers e) which may be mentioned are: products, as are marketed, for example, by Clariant under the trademark Mowiol®.

Examples of suitable polymers f) which may be mentioned are: hydroxypropyl methylcelluloses, as are marketed, for example, by Shin Etsu under the trademark Pharmacoat®.

Examples of polymers g) are the products of BASF Aktiengesellschaft marketed under the trademark Kollicoat® IR.

Of course, the inventive enzyme granules, in addition to the hydrophobic coating, can also have one or more, for example, 1, 2 or 3, further coatings which comprise other materials, for example the coatings taught in the prior art. It is essential to the invention that at least one coating consists of the hydrophobic materials, this layer being able to be arranged as desired and, in particular, arranged directly on the enzyme-comprising core. There is in addition the possibility that the at least one layer is a salt layer or a layer which comprises at least 30% salt. Such a salt layer will preferably be arranged between the core and the outermost layer. The salts mentioned above can be mentioned here as example.

The inventive enzyme granules can be produced by analogy with known production methods for coated enzyme granules, for example analogously to the procedures described in WO 01/00042, WO 03/059086 or PCT/EP 2005/000826.

The inventive enzyme granules advantageously have a mean particle size (particle diameter) in the range from 100 to 2000 μm, in particular in the range from 200 to 1500 μm, and especially in the range from 300 to 1000 μm. The geometry of the granule particles is generally cylindrical having a ratio of diameter to length from about 1:1.3 to 1:3 and with ends rounded if appropriate. Typically, the particle sizes of the inventive coated enzyme granules correspond to those of the uncoated cores which hereinafter are also termed raw granules, that is the ratio of mean particle diameter of the inventive granules to the mean particle diameter of the raw granules will generally not exceed a value of 1.1:1, and in particular a value of 1.09:1.

The inventive phytase-comprising enzyme granules preferably have a phytase activity in the range from 1×10³ to 1×10⁵ FTU, in particular 5×10³ to 5×10⁴ FTU, and especially 1×10⁴ to 3×10⁴ FTU. 1 FTU of phytase activity is thereby defined as the amount of enzyme which liberates 1 micromol of inorganic phosphate per minute from 0.0051 mol/l aqueous sodium phytate at pH 5.5 and 37° C. The phytase activity can be determined, for example as specified in “Determination of Phytase Activity in Feed by a Colorimetric Enzymatic Method”: Collaborative Interlaboratory Study Engelen et al.: Journal of AOAC International Vol. 84, No. 3, 2001, but also Simple and Rapid Determination of Phytase Activity, Engelen et al., Journal of AOAC International, Vol. 77, No. 3, 1994.

The inventive enzyme granules can be produced by analogy with known production methods for coated enzyme granules, for example by analogy with the procedures described in WO 01/00042, WO 03/059086 or PCT/EP 2005/000826.

According to a preferred embodiment, the method comprises the following steps:
a) providing uncoated, phytase-comprising raw granules which comprise at least one phytase and at least one solid carrier material suitable for feeds and which, on suspension or dissolution, after if appropriate required grinding in water, give at 25° C. a pH in the range from 4.5 to 6.5, preferably in the range from 4.6 to 6.0, and particularly preferably in the range from 4.7 to 5.5, and which generally have a water content below 15% by weight, frequently in the range from 1 to 12% by weight, in particular in the range from 3 to 10% by weight, and especially in the range from 5 to 9% by weight, based on the weight of the enzyme-comprising raw granules, and
b) coating the raw granules.

The raw granules can be produced in principle in any desired manner. For example, a mixture comprising the feed-compatible carrier, at least one water-soluble, neutral cellulose derivative, and at least one enzyme and if appropriate further components such as water, buffer, stabilizing metal salts, can be processed to form raw granules in a manner known per se by extrusion, mixer-granulation, fluidized-bed granulation, disk agglomeration or compacting.

In a preferred embodiment, production of the raw granules comprises in a first step the extrusion of a water-comprising dough which comprises at least one water-soluble, neutral cellulose derivative and at least one enzyme and if appropriate further components such as water, buffer, stabilizing metal salts in the amounts stated above. Generally, production of the dough comprises setting the pH in such a manner that the dough, on suspension in water, has a pH in the range from 4.5 to 6.5, preferably in the range from 4.6 to 6.0, and particularly preferably in the range from 4.7 to 5.5. The pH can be set by adding a buffer or a base to the dough. Preferably, the pH of the dough is set in such a manner that the dough is produced using an aqueous enzyme concentrate whose pH on dilution is in the range from 4.5 to 6.5, more preferably in the range from 4.6 to 6.0, and particularly preferably in the range from 4.7 to 5.5. Since the enzyme concentrate frequently has a slightly acidic pH below 4, preferably a buffer or a base will be added. Suitable bases are, in addition to ammonia, ammonia water and ammonium hydroxide, hydroxides, citrates, acetates, formates, carbonates and hydrogencarbonates of alkali metals and alkaline earth metals, and also amines and alkaline earth metal oxides such as CaO and MgO. Examples of inorganic buffering agents are alkali metal hydrogenphosphates, in particular sodium and potassium hydrogenphosphates, for example K₂HPO₄, KH₂PO₄ and Na₂HPO₄. A preferred agent for setting the pH is ammonia or ammonia water, NaOH, KOH. Suitable buffers are, for example, mixtures of aforesaid bases with organic acids such as acetic acid, formic acid, citric acid.

The carrier material generally makes up 50 to 96.9% by weight, preferably 55 to 94.8% by weight, and in particular 60 to 89.7% by weight of the nonaqueous components of the dough. The at least one, water-soluble, neutral cellulose derivative generally makes up 0.1 to 10% by weight, preferably 0.15 to 5% by weight, in particular 0.2 to 2% by weight, and especially 0.3 to 1% by weight, of the nonaqueous components of the dough. The at least one enzyme generally makes up 3 to 49.9% by weight, in particular 5 to 49.8% by weight, and especially 10 to 39.7% by weight, of the nonaqueous components of the dough. The fraction of other components corresponds to the weight fractions given above for the composition of the core.

In addition to aforesaid components, the dough comprises water in an amount which ensures sufficient homogenization for the dough-forming components and adequate consistency (plasticization) of the dough for extrusion. The amount of water required for this can be determined in a manner known per se by those skilled in the art in the field of enzyme formulation. The water fraction in the dough is typically in the range from >15 to 50% by weight, in particular in the range from 20 to 45% by weight, and especially in the range from 25 to 40% by weight, based on the total weight of the dough.

The dough is produced in a manner known per se by mixing the dough-forming components in a suitable mixing apparatus, for example in a conventional mixer or kneader. For this, the solid or solids, for example the carrier material, are intensively mixed with the liquid phase, for example water, an aqueous binder solution, or an aqueous enzyme concentrate. Generally, the carrier will be introduced as solid into the mixer and mixed with an aqueous enzyme concentrate and also with the water-soluble polymer, preferably in the form of a separate aqueous solution or dissolved in the aqueous enzyme concentrate, and also if appropriate with the stabilizing salt, preferably in the form of a separate aqueous solution or suspension, in particular dissolved or suspended in the aqueous enzyme concentrate. If appropriate, further water will be added to set the desired consistency of the dough. Preferably, during mixing, a temperature of 60° C., in particular 40° C., will not be exceeded. Particularly preferably, the temperature of the dough during mixing is 10 to 30° C. If appropriate, therefore, the mixing apparatus will be cooled during dough production.

The resultant dough is subsequently subjected to an extrusion, preferably an extrusion at low pressure. The extrusion, in particular extrusion at low pressure, generally proceeds in an apparatus in which the mix (dough) to be extruded is forced through a matrix. The hole diameter of the matrix determines the particle diameter and is generally in the range from 0.3 to 2 mm, and in particular in the range from 0.4 to 1.0 mm. Suitable extruders are, for example, dome extruders or basket extruders which, inter alia, are marketed by companies such as Fitzpatrick or Bepex. For correct consistency of the mix to be granulated, in this case only a low temperature increase results on passing through the matrix (up to approximately 20° C.). Preferably, the extrusion proceeds under temperature control, that is the temperature of the dough should not exceed a temperature of 70° C., in particular 60° C., during extrusion. In particular, the temperature of the dough during extrusion is in the range from 20 to 50° C.

The extruded dough strands leaving the extruder break up into short granule-like particles or can be broken if appropriate using suitable cutting apparatuses. The resultant granule particles typically have a homogeneous particle size, that is a narrow particle size distribution.

In this manner raw granules are obtained having a comparatively high water content which is generally greater than 15% by weight, for example in the range from 15 to 50% by weight, in particular in the range from 20 to 45% by weight, based on the total weight of the moist raw granules. According to the invention, therefore, before coating, drying is carried out in such a manner that the water content of the raw granules is no greater than 15% by weight and preferably is in the range from 1 to 12% by weight, in particular in the range from 3 to 10% by weight, and especially in the range from 5 to 9% by weight.

The final processing therefore generally comprises a drying step. This preferably proceeds in a fluidized-bed dryer. In this case, a heated gas, generally air or a nitrogen gas stream, is passed from below through the product layer. The gas rate is customarily set so that the particles are fluidized and swirl. As a result of the gas/particles heat transfer, the water evaporates. Since enzyme-comprising raw granules are generally heat-labile, it is necessary to ensure that the temperature of the raw granules does not rise too high, that is generally not above 80° C., and preferably not above 70° C. In particular, the temperature of the granules during drying is in the range from 30 to 70° C. The drying temperature can be controlled in a simple manner via the temperature of the gas stream. The temperature of the gas stream is typically in the range from 140 to 40° C., and in particular in the range from 120 to 60° C. Drying can proceed continuously or batchwise.

After drying, the granules can be further fractionated by means of a sieve (optional). Coarse material and fines can be ground and returned to the mixer for pasting the granulation mix.

In addition, it has proved to be advantageous to round, that is to say spheronize, the still-moist raw granules before carrying out drying. In this case, in particular, the formation of unwanted dust fractions in the end product is decreased.

Apparatuses suitable for rounding the moist raw granules are what are termed spheronizers which essentially have a horizontally rotating disk on which the small extruded rods are forced to the wall by the centrifugal force. The small extruded rods break up on the micronotches prefixed by the extrusion process, so that cylindrical particles are formed having a ratio of diameter to length of about 1:1.3 to 1:3. As a result of the mechanical load in the spheronizer, the initially cylindrical particles are somewhat rounded.

The raw granules obtained after final processing advantageously have a median particle size in the range from 100 to 2000 μm, in particular in the range from 200 to 1500 μm, and especially in the range from 300 to 1000 μm. The median particle size distribution can be determined in a manner known per se by light scattering, for example using a Mastersizer S from Malvern Instruments GmbH or by sieve analysis, for example using a Vibro VS 10000 sieving machine from Retsch. The median particle size is taken by those skilled in the art to mean the D₅₀ value of the particle size distribution curve, that is to say the value which 50% by weight of all particles fall above or below. Preference is given to raw granules having a narrow particle size distribution.

According to a further embodiment, the raw granules are produced by spray drying, spray granulation, spray agglomeration, compacting, granulation in a high-shear mixer or similar apparatuses and methods in which mechanical energy is introduced in the form of agitated parts and/or the introduction of a gas stream and thus particle buildup or raw granule production is performed.

A further alternative is raw granule production by absorption. Here, the enzyme concentrate or an enzyme solution is brought into contact with a carrier material (by adding or spraying on the solution). Carrier materials which come into consideration are the carrier materials mentioned above. The enzyme diffuses together with the solvent present in the solution, preferably water, partially or completely into the carrier material in this method. The solvent, preferably water, can subsequently or in parallel be removed by thermal methods. This can be performed, for example, in a fluidized bed, a fluidized-bed dryer or other dryers.

Subsequently, the resultant raw granules are coated. For this, in a manner known per se, one of the aforesaid coating materials is applied to the raw granules. The coating-forming material can be applied in a manner known per se by application of a solution, dispersion or suspension of the coating-forming material in a suitable solvent, for example water, or by application of a melt of the material. The application of a melt is preferred according to the invention, because the subsequent removal of solvent or dispersion medium can thereby be avoided. This means that for application of a melt, the use of an expensive dryer/coater (for example a fluidized-bed dryer) is not required, but the use of a mixer is possible. Coating with a melt of the material is also termed hereinafter melt coating.

Suitable methods for applying the coating comprise coating in a fluidized bed, and also coating in a mixer (continuously or batchwise), for example in a granulation drum, a ploughshare mixer, for example from Lodige, a paddle mixer, for example from Forberg, a Nauta mixer, a granulating mixer, a granulating dryer, a vacuum coater, for example from Forberg, or a high-shear granulator.

In particular, the raw granules are coated
i) in a fluidized bed, for example by spraying the raw granules with a melt, a solution or dispersion of the material forming the coating; and also
ii) in one of the abovementioned mixing apparatuses by introduction of the raw granules into a melt of the material forming the coating or by spraying or dousing the raw granules with a melt, a solution or dispersion of the material forming the coating.

Coating the raw granules by spraying with a melt, a solution or dispersion in a fluidized bed is particularly preferred according to the invention. Spraying the raw granules with a melt, a solution or dispersion of the material can be carried out in the fluidized-bed apparatus in principle in the bottom-spray method (nozzle is seated in the gas-distribution plate and sprays upwards) or in the top-spray method (coating is sprayed into the fluidized bed from the top).

The raw granules can be coated in the context of the inventive method continuously or batchwise.

According to a first preferred embodiment of the inventive method, the raw granules are charged into a fluidized bed, swirled and, by spraying on an aqueous or nonaqueous, preferably aqueous, dispersion of the material forming the coating, are coated with this material. For this use is made of preferably a liquid which is as highly concentrated as possible and still sprayable, such as, for example, a 10 to 50% strength by weight aqueous dispersion or nonaqueous solution or dispersion of the material.

The solution or dispersion of the material is preferably sprayed on in such a manner that the raw granules are charged into a fluidized-bed apparatus or a mixer and sprayed onto the spray material with simultaneous heating of the charge. The energy is supplied in the fluidized-bed apparatus by contact with heated drying gas, frequently air. Preheating the solution or dispersion can be expedient when as a result spray material having a higher dry substance fraction can be sprayed. When use is made of organic liquid phases, solvent recovery is expedient and the use of nitrogen as drying gas to avoid explosive gas mixtures is preferred. The product temperature during coating should be in the range from about 30 to 80° C., and in particular in the range from 35 to 70° C., and especially in the range from 40 to 60° C. Coating can be carried out in the fluidized-bed apparatus in principle in the bottom-spray method (nozzle is seated in the gas-distribution plate and sprays upwards) or in the top-spray method (coating is sprayed into the fluidized bed from the top). When a mixer is used for coating, after the solution or dispersion is sprayed on, the solvent or the liquid of the dispersion must be removed. This can be carried out in a dryer.

According to a second, particularly preferred embodiment of the inventive method, the raw granules charged into a fluidized bed or mixer are coated with a melt of the material forming the coating. Melt coating in a fluidized bed is preferably carried out in such a manner that the raw granules to be coated are charged into the fluidized-bed apparatus. The material intended for the coating is melted in an external reservoir and pumped, for example via a heatable line to the spraying nozzle. Heating the nozzle gas is expedient. Spraying rate and inlet temperature of the melt are preferably set in such a manner that the material still runs readily on the surface of the granules and evenly coats them. Preheating the granules before spraying the melt is possible. In the case of materials having a high melting point, generally the temperature will be selected in such a manner that a loss of enzyme activity is substantially avoided. The product temperature should therefore preferably be in the range from about 30 to 80° C., and in particular in the range from 35 to 70° C., and especially in the range from 40 to 60° C. Melt coating can also be carried out in principle by the bottom-spray method or by the top-spray method.

Melt coating can be carried out in a mixer in two different ways. Either the granules to be coated are charged into a suitable mixer and a melt of the material is sprayed or poured into the mixer. Another possibility is to mix the hydrophobic material present in solid form with the product. By supplying energy via the vessel wall or via the mixing tools, the hydrophobic material is melted and thus coats the raw granules. According to requirement, from time to time a little release agent can be added. Suitable release agents are, for example, silicic acid, talcum, stearates and tricalcium phosphate, or salts such as magnesium sulfate, sodium sulfate or calcium carbonate.

The solutions, dispersions or melts used for coating can, if appropriate, be admixed with other additives, such as, for example, microcrystalline cellulose, talcum and kaolin, or salts.

In a particular inventive embodiment of the method, the addition of release agents during application of the material or the addition of release agents to the solution, dispersion or melt to be applied can be omitted. This is possible, in particular, when the enzyme cores used have median particle sizes of at least 300 μm, preferably at least 350 μm, in particular at least 400 μm, for example in the range from 250 to 1600 μm, preferably in the range from 300 μm to 1500 μm, and in particular in the range from 400 μm to 1400 μm, and simultaneously the amount of coating material used based on the total particle is no greater than 30% by weight, preferably no greater than 25% by weight, in particular no greater than 20% by weight, and especially no greater than 17% by weight. In these cases, enzyme cores may be coated particularly readily without agglomeration of the particles.

The addition of a flow aid after the coating step can enhance the flow properties of the product. Typical flow aids are silicic acids, for example the Sipernat products from Degussa or the Tixosil products from Rhodia, talcum, stearates and tricalcium phosphate, or salts such as magnesium sulfate, sodium sulfate or calcium carbonate. The flow aids are added to the coated product in an amount of from 0.005% by weight to 5% by weight based on the total weight of the product. Preferred contents are 0.1% by weight to 3% by weight, and particularly preferred 0.2% by weight to 1.5% by weight.

The invention further relates to feed compositions, and in particular pelleted feed compositions which, in addition to customary components, comprise at least one feed additive in accordance with the above definition as admixture.

Finally, the invention also relates to the use of a feed additive according to the above definition for producing feed compositions, in particular hydrothermally treated, and especially pelleted, feed compositions.

For production of the feed compositions, the coated enzyme granules produced according to the invention are mixed with conventional animal feed (such as, for example, pig-fattening feed, piglet feed, sow feed, broiler feed and turkey feed). The enzyme granule fraction is selected in such a way that the enzyme content is, for example, in the range from 10 to 1000 ppm. Subsequently, the feed is pelleted using a suitable pellet press. For this the feed mixture is customarily conditioned by steam introduction and subsequently pressed through a matrix. Depending on the matrix, pellets of about 2 to 8 mm in diameter can be produced in this way. The highest process temperature occurs in this case during conditioning or during pressing of the mixture through the matrix. Here, temperatures in the range from about 60 to 100° C. can be reached.

### COMPARATIVE EXAMPLE C1

a) In an aqueous phytase concentrate having a dry mass content of about 25 to 35% by weight, a pH in the range of 3.7-3.9 and an activity of 26 000 to 36 000 FTU/g, 1% by weight of zinc sulfate hexahydrate, based on the concentrate, was dissolved at 4-10° C.
b) In a mixer having a chopper blade, 900 g of corn starch were charged, homogenized and to this were added slowly at temperatures of 10 to 30° C. with homogenization simultaneously 380 g of the zinc sulfate-comprising phytase concentrate and 140 g of a 10% strength by weight solution of polyvinyl alcohol (degree of hydrolysis: 87-89%). The mixture was homogenized with cooling of the mixer for a further 5 min at temperatures in the range from 10 to 50° C., then the resultant dough was transferred to a dome extruder and the dough was extruded at temperatures in the range from 30 to 50° C. through a matrix having an orifice diameter of 0.7 mm to give 5 cm long strands.
c) The resultant extrudate was rounded in a rounding machine (type P50, from Glatt) for 5 min. at 350 min⁻¹ (speed of rotation of the rotating disk) and then dried to a residual moisture of about 6% by weight in a fluidized-bed dryer at a temperature of up to 40° C. (product temperature).

The resultant raw granules had an activity of approximately 14 200 FTU/g. The granules had a particle size of a maximum of 1300 μm and median particle size of 650 μm (sieve analysis).
d) For the subsequent coating, the raw granules were charged into a laboratory fluidized bed Aeromat type MP-1 from Niro-Aeromatic. As receiving vessel, use was made of a plastic cone having a gas-distribution plate diameter of 110 mm and a perforated plate having 12% open area. The coating agent was a commercially available triglyceride based on saturated C₁₆/C₁₈ fatty acids (melting point 57-61° C., iodine value 0.35, saponificaton value 192).

The raw granules (700 g) charged into the fluidized bed were heated to a product temperature of 45° C. with swirling using an air rate of 50 m³/h. 124 g of the triglyceride were melted in a glass beaker at 85° C. and sprayed onto the raw granules by means of a two-fluid nozzle (1 mm) in the bottom-spray method by reduced-pressure suction at 1 bar spraying pressure using heated spraying gas from 80 to 90° C. During spraying, the coating material and the intake line were heated to 80 to 90° C. in order to obtain a fine spray mist so that an even coating layer formed on the particles and completely enveloped them. During the spray process, the air rate was increased to 60 m³/h, in order to maintain the fluidized-bed height. The spray time was 15 min, the product temperature being 45 to 48° C. and the feed air temperature approximately 45° C. Subsequently, the product was cooled with swirling to 30° C. at 50 m³/h feed air.

A product was obtained having the following characteristic data:

| Composition: | | |
|---|---|---|
| Corn starch | 68.0% | by weight |
| Phytase (dry mass) | 12.0% | by weight |
| Polyvinyl alcohol: | 1.1% | by weight |
| Zinc sulfate (ZnSO₄): | 0.4% | by weight |
| Triglyceride: | 15.0% | by weight |
| Residual moisture: | 3.5% | by weight |

| | |
|---|---|
| Phytase activity: | approximately 11 800 FTU/g |
| Appearance (microscope): | particles having a smooth surface |

### EXAMPLE 1

a) In an aqueous phytase concentrate having a dry mass content of about 25-35% by weight, a pH in the range of 3.7-3.9 and an activity of 26 000-36 000 FTU/g, 1% by weight of zinc sulfate hexahydrate, based on the concentrate, was dissolved at 4-10° C. Subsequently, a pH of 5 was set by adding 5% by weight, based on the phytase concentrate, of a 5% strength by weight aqueous ammonia solution.
b)+c) Using the neutralized phytase concentrate produced in a), rounded raw granules were produced according to the protocol of comparative example 1, steps b) and c).

The resultant raw granules had an activity of approximately 13 300 FTU/g. The granules had a particle size of a maximum of 1300 μm and a median particle size of 645 μm (sieve analysis).
d) The resultant raw granules were then coated according to the protocol of comparative example C1, step d) in a laboratory fluidized bed Aeromat type MP-1 from Niro-Aeromatic. The coating agent was a commercially available triglyceride based on saturated C₁₆/C₁₈-fatty acids (melting point 57-61° C., iodine value 0.35, saponification value 192).

A product was obtained having the following characteristic data:

| Composition: | | |
|---|---|---|
| Corn starch | 68.0% | by weight |
| Phytase (dry mass) | 12.0% | by weight |
| Polyvinyl alcohol: | 1.1% | by weight |
| Zinc sulfate (ZnSO₄): | 0.4% | by weight |
| Triglyceride: | 15.0% | by weight |
| Residual moisture: | 3.5% | by weight |

| | |
|---|---|
| Phytase activity: | approximately 11 050 FTU/g |
| Appearance (microscope): | particles having a smooth surface |

### EXAMPLE 2

a) In an aqueous phytase concentrate having a dry mass content of about 25-35% by weight, a pH in the range of 3.7-3.9 and an activity of 26 000-36 000 FTU/g, 1% by weight of zinc sulfate hexahydrate, based on the concentrate, was dissolved at 4-10° C. The concentrate was heated to 30° C. and 1.1% by weight of methylcellulose (molecular weight of 70 000-120 000 g/mol, viscosity: 4600 cps at 2% by weight in water and 20° C., degree of substitution 1.6-1.9) was added to this and the mixture was stirred until the methylcellulose had completely dissolved. Then, a pH of 5 was set by adding 5% by weight, based on the phytase concentrate, of a 5% strength by weight aqueous ammonia solution.
b) In a mixer having a chopper blade, 890 g of corn starch were charged, homogenized, and to this were added 433 g of the phytase concentrate from step a) slowly at temperatures of 10 to 30° C. with homogenization. The mixture was homogenized with cooling of the mixer for a further 5 min at temperatures in the range from 10 to 50° C., then the resultant dough was transferred to a dome extruder and the dough was extruded at temperatures in the range from 30 to 50° C. through a die having an orifice diameter of 0.7 mm to give 5 cm long strands.
c) The resultant extrudate was rounded in a rounding machine (type P50, from Glatt) for 5 min at 350 min⁻¹ (speed of rotation of the disk) and subsequently dried in a fluidized-bed dryer at a temperature of 40° C. (product temperature) to a residual moisture of about 6% by weight.

The resultant raw granules had an activity of approximately 12 700 FTU/g. The granules had a particle size of a maximum of 1400 μm and a median particle size of 662 μm (sieve analysis).
d) The resultant raw granules were then coated in accordance with the protocol from comparative example C1, step b) in a laboratory fluidized bed Aeromat type MP-1 from Niro-Aeromatic. The coating agent was a commercially available triglyceride based on saturated C₁₈/C₁₈-fatty acids (melting point 57-61° C., iodine value 0.35, saponification value 192).

A product was obtained having the following characteristic data:

| Composition: | | |
|---|---|---|
| Corn starch | 68.6% | by weight |
| Phytase (dry mass) | 12.0% | by weight |
| Methylcellulose: | 0.5% | by weight |
| Zinc sulfate (ZnSO₄): | 0.4% | by weight |
| Triglyceride: | 15.0% | by weight |
| Residual moisture: | 3.5% | by weight |

| | |
|---|---|
| Phytase activity: | approximately 10 450 FTU/g |
| Appearance (microscope): | Particles having a smooth surface |

### COMPARATIVE EXAMPLE C2

Production was performed analogously to example 2, but in contrast to the protocol specified there, no aqueous ammonia solution was added.

A product having the following characteristic data was obtained:

| Composition: | | |
|---|---|---|
| Corn starch | 68.6% | by weight |
| Phytase (dry mass) | 12.0% | by weight |
| Methylcellulose: | 0.5% | by weight |
| Zinc sulfate (ZnSO₄): | 0.4% | by weight |
| Triglyceride: | 15.0% | by weight |
| Residual moisture: | 3.5% | by weight |

| | |
|---|---|
| Phytase activity: | approximately 10 760 FTU/g |
| Appearance (microscope): | Particles having a smooth surface |

### Experiment 1: Determination of Pelleting Stability

To assess the pelleting stability of the above-described enzyme granules, a standard pelleting was established. For this, to improve the analytical content determinations, the dosage in the feed is increased. The pelleting is carried out in such a manner that a conditioning temperature of 80 to 85° C. is achieved. Representative samples of the feed before and after pelleting are obtained. The enzyme activity is determined in these samples. If appropriate after correcting for the content of enzyme which is present in the native state, the losses due to pelleting and the relative residual activity (=retention) can be calculated.

The analytical method for phytase is described in various publications: Simple and Rapid Determination of Phytase Activity, Engelen et al., Journal of AOAC International, Vol. 77, No. 3, 1994; Phytase Activity, General Tests and Assays, Food Chemicals Codex (FCC), IV, 1996, p. 808-810; Bestimmung der Phytaseaktivitat in Enzymstandardmaterialien und Enzympraparaten [Determination of phytase activity in standard enzyme materials and enzyme preparations] VDLUFA-Methodenbuch [Handbook of Methods of the German Association of Agricultural Analytical and Research Institutes], Volume III, 4th supplement 1997; or Bestimmung der Phytaseaktivitat in Futtermitteln und Vormischungen [Determination of phytase activity in feeds and premixes] VDLUFA-Methodenbuch, Volume III, 4th supplement 1997. As feed, use was made of a conventional broiler feed having the following composition:

| | |
|---|---|
| Corn | 45.5% |
| Soybean extraction meal | 27.0% |
| Full-fat soybeans | 10.0% |
| Peas | 5.0% |
| Tapioca | 4.7% |
| Soybean oil | 3.5% |
| Lime | 1.35% |
| Monocalcium phosphate | 1.30% |
| Cattle salt | 0.35% |
| Vitamin/trace element premix | 1.00% |
| D,L-Methionine | 0.25% |
| L-Lysine HCl | 0.05% |
| | 100% |

The coated granules produced in the above examples were mixed with the above standard feed (content 500 ppm), pelleted and the samples obtained were analyzed. The relative improvement in retention of enzyme activity compared with the granules from comparative example C1 was calculated as follows: Ratio of retention of enzyme activity of the improved granules to retention of enzyme activity of the granules from comparative example C1. The results are summarized in table 1 hereinafter.

TABLE 1
Pelleting stability achieved

| | Description of the granules | Relative pelleting |
|---|---|---|
| No. | (coating, binder, pH) | stability [%] |
| Comparative | 15% fat, 1.1% PVA, pH 3.9 | 100 |
| example C1 | | |
| Example 1 | 15% fat, 1.1% PVA, pH 5 | 120 |
| Example 2 | 15% fat, 0.5% MC, pH 5 | 123 |
| Comparative | 15% fat, 0.5% MC, pH 3.9 | 112 |
| example C2 | | |

PVA = Polyvinyl alcohol
MC = Methylcellulose

### Experiment 2:

To assess the pH of the granules from comparative example C1, 200 ml of demineralized water were charged into a 250 ml conical flask and stirred at 25° C. using a magnetic stirrer of type IKA RCT basic at stage 7-8. During stirring, 5 g of the granules from comparative example C1 comminuted in a mortar were slowly added and the pH was measured. The granules first largely floated undissolved at the top. After approximately 5 minutes, the pH first fell to 6.2 and the granules were for the most part dissolved. After 13 min, the granules were virtually completely dissolved except for fines and the pH was between 4.28 and 4.35. A turbid milky suspension was formed. After 25 min, the experiment was stopped since no changes in pH were any longer observed.

### Experiment 3:

To assess the pH of the granules from example 1, 200 ml of demineralized water were charged into a 250 ml conical flask and stirred using a magnetic stirrer of type IKA RCT basic at stage 7-8 at 25° C. During stirring, 5 g of the granules from comparative example C1 comminuted in a mortar were slowly added and the pH was measured. The granules first floated for the most part undissolved at the top. After approximately 5 minutes, the pH fell to 6.5 and the granules were for the most part dissolved. After approximately 15 min, the granules were virtually completely dissolved except for fines and the pH was between 5.6 and 5.7. A turbid milky suspension was formed. After 30 min the experiment was stopped, since no changes in pH were any longer observed.

1. A phytase-comprising enzyme granule for feeds, comprising one or more particles of which have
a) a core which comprises at least one phytase and at least one solid carrier material suitable for feeds, and
b) a coating arranged on the core,
the cores of the particles or the entire particle, on suspension or dissolution in demineralized water at 25° C. giving a pH in the range from 4.5 to 6.5.

2. The enzyme granule according to claim 1, wherein the core comprises at least one buffer to set the pH.

3. The enzyme granule according to claim 1, wherein at least 70% by weight of the at least one coating arranged on the surface of the core comprises substances selected from waxes, saturated fatty acids, esters of saturated fatty acids, polyolefins, cellulose derivatives, polyvinyl acetate, polyvinylpyrrolidone, polyvinyl alcohol, vinyl acetate/vinylpyrrolidone copolymers, polyalkylene glycols or mixtures thereof.

4. The enzyme granule according to claim 1, wherein at least 90% by weight of the at least one coating arranged on the surface of the core comprises a water-insoluble hydrophobic substance.

5. The enzyme granule according to claim 4, wherein the hydrophobic substance is selected from one or more triglycerides.

6. The enzyme granule according to claim 1, wherein the weight ratio of core to coating is in the range from 70:30 to 99:1.

7. The enzyme granule according to claim 1, wherein the particles have a mean particle size in the range from 100 to 2000 μm.

8. The enzyme granule according to claim 1, wherein the enzyme core is produced using an aqueous enzyme concentrate which, at 25° C., has a pH in the range from 4.5 to 6.5.

9. The enzyme granule according to claim 1, wherein the carrier material comprises at least one water-insoluble polymeric carbohydrate.

10. The enzyme granule according to claim 1, wherein the enzyme core comprises
i) 50 to 96.9% by weight of at least one solid carrier material suitable for feeds,
ii) 0.1 to 20% by weight of at least one water-soluble polymeric binder,
iii) 3 to 49.9% by weight of at least one phytase or an enzyme mixture which comprises at least one phytase, and
iv) if appropriate water,
the weight fractions of i), ii) and iii) each being based on the nonaqueous components of the core.

11. The enzyme granule according to claim 10, wherein the water-soluble polymeric binder is selected from polyvinyl alcohol or water-soluble polysaccharides.

12. The enzyme granule according to claim 11, wherein the water-soluble polymeric binder comprising comprises a neutral cellulose ether.

13. The enzyme granule according to claim 1, wherein the core additionally comprises a salt stabilizing the enzyme in an amount of 0.1 to 10% by weight, based on the total weight of all nonaqueous components of the core.

14. The enzyme granule according to claim 13, wherein the salt is selected from zinc sulfate or magnesium sulfate.

15. A method for producing solid phytase-comprising enzyme granules according to claim 1, comprising:
a) providing uncoated, phytase-comprising raw granules which comprise at least one phytase and at least one solid carrier material suitable for feeds and which, on suspension or dissolution in demineralized water at 25° C., give a pH in the range from 4.5 to 6.5, and
b) coating the raw granules.

16. The method according to claim 15, wherein the phytase-comprising raw granules are produced using a phytase-comprising aqueous enzyme concentrate, the pH of which in demineralized water at 25° C. being in the range from 4.5 to 6.5.

17. The method according to claim 16, wherein in step a), a mixture comprising the feed-compatible carrier and the phytase-comprising aqueous enzyme concentrate is processed to form raw granules by extrusion, mixer-granulation, fluidized-bed granulation, disk agglomeration, spray agglomeration, spray granulation or compacting.

18. The method according to claim 17, wherein step a) comprises the extrusion of an enzyme-comprising dough which, in addition to water
i) comprises 50 to 96.9% by weight of at least one solid carrier material suitable for feeds,
ii) 0.1 to 20% by weight of at least one water-soluble neutral cellulose derivative,
iii) 3 to 49.1% by weight of at least one enzyme, the weight fractions of i), ii) and iii) in each case being based on the nonaqueous components of the dough.

19. The method according to claim 15, wherein in step b), the material used for producing the coating is used in the form of its melt.

20. The method according to claim 15, wherein in step a), the raw granules, before coating, are subjected to spheronization and subsequently dried to a residual water content of no greater than 15% by weight, based on the total weight of the raw granules.

21. A phytase-comprising raw granule comprising at least one phytase and at least one solid carrier material suitable for feeds which, on suspension or dissolution in demineralized water at 25° C., gives a pH in the range from 4.5 to 6.5.

22. A method of producing feed comprising utilizing the phytase-comprising enzyme granule according to claim 1 in feeds.

23. A feed comprising at least one phytase-comprising enzyme granule according to claim 1 and customary feed components.

24. The feed according to claim 23 in the form of a pelleted feed.

25. The enzyme granule of claim 12, wherein the neutral cellulose ether is methylcellulose.
